(19) United States
(12) Patent Application Publication
(10) Pub. No.: US 2020/0405621 A1
(43) Pub. Date: Dec. 31, 2020

(54) Skin Care Composition

(71) Applicant: The Procter & Gamble Company, Cincinnati, OH (US)

(72) Inventors: Tomohiro HAKOZAKI, Cincinnati, OH (US); Bin FANG DEYER, Loveland, OH (US)

(21) Appl. No.: 16/455,966

(22) Filed: Jun. 28, 2019

(51) Int. Cl. A61K 8/9789; A61K 8/81; A61Q 19/02

(52) U.S. Cl. CPC A61K 8/9789; A61Q 19/02; A61K 8/8147

(57) ABSTRACT

Methods and compositions for improving the appearance of skin are provided. The methods and compositions are especially suited for improving sallow-looking skin by enhancing bilirubin breakdown in skin. The methods and compositions herein utilize a water lily extract that demonstrates a surprising ability to reduce bilirubin levels. The method involves applying a skin care composition containing an effective amount of water lily extract to a target portion of skin where a reduction in bilirubin level is desired.

### FIELD

The present invention is directed generally to skin care compositions for treating skin tone conditions. More specifically, the present invention is directed to a method that utilizes an effective amount of a water lily extract to improve skin tone appearance. Even more specifically, the compositions and methods disclosed herein provide an unexpected bilirubin degradation benefit.

### BACKGROUND

Sallow or yellow-looking skin is commonly associated with poor health or old age. It is known that sallow-looking skin can be caused by the buildup of a yellow pigment known as bilirubin. Bilirubin is produced as a result of the catabolic breakdown of heme in red blood cells, when old or damaged cells are cleared from the body as part of the normal process of clearing waste from the blood. Typically, bilirubin is processed by the liver and then excreted from the body as waste. However, in some instances, bilirubin can collect in skin tissue resulting in a yellow or sallow appearance. For example, bilirubin is responsible for the yellowish coloring in skin associated with bruising or jaundice. Thus, it would be desirable to provide a way to improve the appearance of yellow or sallow looking skin associated with bilirubin buildup.

One known method of reducing bilirubin levels in skin is through light therapy (a.k.a. photo therapy). Certain wavelengths of light react with bilirubin and convert it to a form that is more easily processed and removed by the body. Indeed, light therapy is one of the most common forms of treating undesirably high bilirubin levels in newborn babies (i.e., hyperbilirubinemia). However, light therapy can require spending a significant amount of time (e.g., 12-72 hours) under an artificial light source in an away-from-home environment (e.g., hospital), which may be undesirable for many people suffering from such conditions. Accordingly, it would be desirable to provide a more convenient method of degrading bilirubin in skin to improve the appearance of a sallow-looking skin.

Cosmetic compositions claiming to improve the degradation of bilirubin are commercially available, but many, if not all, such products are intended for use in improving the appearance of undereye dark circles. For example, Eyedeline™ marine ingredient brand cosmetic eye care product from Lipotec purports to improve the appearance of undereye dark circles by enhancing bilirubin degradation, among other things. Truthinaging.com discloses that cosmetic and beauty products comprising N-hydroxysuccinimide, such as the eye serum product available from AQ Skin Solution, activate the elimination of blood originated pigments such as bilirubin, which contribute to the appearance of undereye dark circles. In another example, a botanical ingredient obtained from the White Bird of Paradise flower (commercially available as Vivillume™ from Lonza, New Jersey) is claimed to degrade bilirubin. Cosmetic products sold by the Avani company (Spain) for treating undereye dark circles are advertised as including Vivillume™. Eye treatment products are formulated to treat the relatively small areas of skin present in the periorbital region of the face, and thus may not be suitable for treating larger areas of skin to address appearance issues associated with the presence of bilirubin in the skin (e.g., sallow-looking skin and/or uneven skin tone). In addition, at least some people who suffer from sallow-looking skin also want a skin care product that treats other cosmetic skin conditions such as fine lines, wrinkles, hyperpigmented spots, and/or dull skin.

Accordingly, it would be desirable to provide a composition that improves the appearance of skin texture and/or tone. It would also be desirable to improve the appearance of sallow-looking skin by applying a composition to skin that improves bilirubin degradation.

### SUMMARY

A method of improving the appearance of skin and/or degrading bilirubin in skin is described herein. The method comprises identifying a target portion of skin on a person where treatment is desired (e.g., a reduction in bilirubin is desired); and applying a composition comprising an effective amount of water lily extract to the target portion of skin during a treatment period, wherein the effective amount of water lily extract reduces bilirubin level.

### BRIEF DESCRIPTION OF THE DRAWINGS

FIG. 1 is a bar chart comparatively illustrating the effect of a suitable water lily extract on bilirubin reduction.

FIG. 2 is line chart illustrating the direct correlation between bilirubin level and b* value.

### DETAILED DESCRIPTION

The drawbacks associated with the presence and/or buildup of bilirubin in skin are well known, but conventional treatments for reducing bilirubin levels in skin, such as light therapy, may not suitable for all users. Prior to the present discovery, it was not commonly known that certain extracts of the water lily plant (genus Nymphaea) can be used to degrade bilirubin. Surprisingly, it has now been discovered that only certain water lily extracts can provide a suitable bilirubin degradation benefit. In addition, the skin care compositions described herein can improve skin appearance by chronically treating other cosmetic skin conditions such as the appearance of wrinkles, rough skin texture and skin dullness.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

In all embodiments, all ingredient percentages are based on the weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges; delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

“About” modifies a particular value by referring to a range equal to plus or minus twenty percent (+/−20%) or less (e.g., less than 15%, 10%, or even less than 5%) of the stated value.

“Apply” or “application”, as used in reference to a composition, means to apply or spread the compositions of the present invention onto a human skin surface such as the epidermis.

“Bilirubin” means the compound identified as CAS No. 635-65-4 and having the chemical formula C₃₃H₃₆N₄O₆ and the following structure:

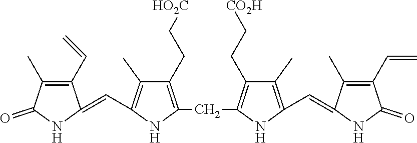 [SMILES: C=CC1=C(C)/C(=C/c2[nH]c(Cc3[nH]c(/C=C4\NC(=O)C(C)=C4C=C)c(C)c3CCC(=O)O)c(CCC(=O)O)c2C)NC1=O]

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration, food additives, and materials used in non-cosmetic consumer products including over-the-counter medications.

“Cosmetic composition” means a composition comprising a cosmetic agent such as the water lily extract described herein. Examples of cosmetic compositions include color cosmetics (e.g., foundations, lipsticks, concealers, and mascaras), skin care compositions (e.g., moisturizers and sunscreens), personal care compositions (e.g., rinse-off and leave on body washes and soaps), hair care compositions (e.g., shampoos and conditioners).

“Effective amount” means an amount of a compound or composition sufficient to significantly induce a positive benefit to keratinous tissue over the course of a treatment period. The positive benefit may be a health, appearance, and/or feel benefit, including, independently or in combination, the benefits disclosed herein. In a specific example, an effective amount of water lily extract is an amount sufficient to reduce bilirubin level.

“Enzymatic hydrolysate” mean an extract obtained (e.g., from Nymphaea alba) using a method comprising at least one enzymatic hydrolysis step.

“Hydrolysate” means an extract obtained (e.g., from Nymphaea alba) by using a method comprising at least one enzymatic or chemical hydrolysis step.

“Improve the appearance of” means providing a measurable, desirable change or benefit in male and/or female skin tone appearance, which may be quantified, for example, by a decrease in b* value of skin. Exemplary methods for determining improvements in appearance are described in more detail below.

“Juice” refers to the liquid expelled from water lily plant material as a result of pressing or other mechanical processing. Juice can contain solid particles, semi-solid particles, and/or droplets of water-immiscible liquids of a variety of sizes (collectively referred to as “water lily particles”) in an aqueous serum.

“L*a*b*” refers to the commonly recognized color space specified by the International Commission on Illumination (“CIE”). The three coordinates represent (i) the lightness of the color (i.e., L*=0 yields black and L*=100 indicates diffuse white), (ii) the position of the color between magenta and green (i.e., negative a* values indicate green while positive a* values indicate magenta) and (iii) the position of the color between yellow and blue (i.e., negative b* values indicate blue and positive b* values indicate yellow).

“Safe and effective amount” means an effective amount of an ingredient that is low enough to avoid serious side effects (within the scope of sound medical judgment).

“Sallow,” when referring to the appearance of skin herein, means an unusual yellow or pale skin tone, with regard to a particular individual, which is commonly associated with an unhealthy state. Sallow-appearing skin can be diagnosed objectively (e.g., with a color value such as L* or b*) or subjectively (e.g., by a skin care professional or via self-diagnosis by a consumer).

“Skin care” means regulating and/or improving a skin condition. Some nonlimiting examples include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin.

“Skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

“Skin care composition” means a composition that includes a skin care active and regulates and/or improves skin condition.

“Skin tone” means the overall appearance of basal skin color or color evenness. Skin tone is typically characterized over a larger area of the skin, which is generally more than 100 mm², up to and including the entirety of the facial skin or other bodily skin surface (e.g., arms, legs, back, hands, neck, chest and abdomen). Skin tone can be measured by image analysis. One measure of skin tone is lightness, which can be measured by the L* coordinate in the L*a*b* color space (International Commission on Illumination). Chromophore mapping such as melanin mapping and melanin concentration may also be used as an indicator of skin tone. Mean melanin may be calculated from the chromophore map data. Additionally, skin tone can be correlated to melanin evenness (e.g., standard deviation) which also may be calculated from the chromophore map data.

“Treatment period,” as used herein, means the length of time and/or frequency that a material or composition is applied to a target skin surface.

Composition

The compositions herein contain an effective amount of water lily extract disposed in a dermatologically acceptable carrier and are intended for topical application to human skin. The amount of water lily extract should be sufficient to demonstrate an in vitro bilirubin degradation benefit and/or improve the appearance of sallow looking skin after a suitable course of treatment (e.g., 2, 4 or 8 weeks). The compositions herein can also treat other skin conditions and may optionally include one or more additional skin actives or other ingredients of the type commonly included in topical skin care compositions. The skin care compositions herein can be made using conventional methods of combining skin ingredients.

The skin care compositions herein may be cosmetic compositions, pharmaceutical compositions, or cosmeceutical compositions, and may be provided in various product forms, including, but not limited to, solutions, suspensions, lotions, creams, gels, toners, sticks, sprays, aerosols, ointments, cleansing liquid washes and solid bars, pastes, foams, mousses, shaving creams, wipes, strips, patches, electrically-powered patches, hydrogels, film-forming products, facial and skin masks (with and without insoluble sheet), make-up such as foundations, eye liners, and eye shadows, and the like. In some instances, the composition form may follow from the particular dermatologically acceptable carrier chosen. For example, the composition (and carrier) may be provided in the form of an emulsion (e.g., water-in-oil, oil-in-water, or water-in-oil-in water) or an aqueous dispersion.

Water Lily Extract

The compositions herein include an effective amount of water lily extract obtained from a plant in the genus Nymphaea. The water lily extract may be provided as a liquid (e.g., an aqueous solution containing water lily plant material or a hydrolysate thereof) or as a solid (e.g., a powder formed by drying the liquid water lily extract and/or a maltodextrin carrier thereof). The water lily extract may be mixed with a suitable carrier prior to incorporation into the cosmetic composition. For example, powdered water lily extract may be mixed with a maltodextrin carrier (e.g., at a ratio of 1:9) or an aqueous carrier (e.g., water and/or a water soluble/miscible material). The amount of water lily extract that is effective can differ from one particular source of extract to another (i.e., supplier variation). However, the effective amount can be determined by the skilled artisan, for example, by measuring the level of bilirubin degradation activity according to the Bilirubin Quantification Assay described in more detail below. As with any extract, the concentration of active components and/or level of activity will depend on factors such as the final dilution volume of the extract product, the particular extraction method employed, the natural range of variation among individual plants, and other common factors known to those skilled in the art.

The water lily extract of the present invention may be obtained from a suitable species of water lily and/or portion of the water lily plant (e.g., flower, root, leaf, stem, seed, juice or a combination of these), as long as the extract demonstrates the ability to suitably degrade bilirubin. Examples of water lily species that may be suitable for use herein include Nymphaea alba, Nymphaea gigantea, Nymphaea tetragona, Nymphaea lotus, Nymphaea caerulea, Nymphaea pubescens, Nymphaea amazonum, Nymphaea ampla, Nymphaea blanda, Nymphaea calliantha, Nymphaea candida, Nymphaea capensis, Nymphaea colorata, Nymphaea conardii, Nymphaea elegans, Nymphaea fennica, Nymphaea flavovirens, Nymphaea gardneriana, Nymphaea glandulifera, Nymphaea heudelotii, Nymphaea jamesoniana, Nymphaea leibergii, Nymphaea macrosperma, Nymphaea Mexicana, Nymphaea micrantha, Nymphaea nouchali, Nymphaea odorata, Nymphaea rubra, Nymphaea rudgeana, Nymphaea stuhmannii, Nymphaea sulfurea, Nymphaea thermarun, and Nymphaea violacea. Extracts obtained from the flower of Nymphaea alba may be particularly suitable for use in the present compositions. Nymphaea alba includes Castalia alba (L.) Greene, Castalia minoriflora Simonk, Castalia speciosa Salisb, Nymphaea occidentalis Moss or Nymphaea minonflora (Simonk.) Wissjul, but does not include species of Lotus or Nelumbo nucifera.

The water lily extract may be in the form of a yellow, odorless, aqueous solution with a dry matter content of between about 10 and 55 g/L (e.g., between 15 and 45 g/L, between 20 and 40 g/L or even between 24 and 35). The active ingredient(s) obtained from water lily extract may contain a carbohydrate, protein or mineral, a hydrolysate of one or more of these or combination thereof. In some instances, the hydrolysate may include an enzymatic hydrolysate, obtained by conducting one or more enzymatic hydrolyses on the water lily extract or components thereof (e.g., carbohydrate or protein). When two more enzymatic hydrolyses are conducted, different types of enzymes may be used (e.g., a carbohydrase and a protease, two different carbohydrases, or two different proteases). The water lily extract may have a carbohydrate fraction of between 20% and 70% (e.g., 23% to 62% or 25% to 55%) by weight, based on the weight of the dry matter. The carbohydrate content can be determined according to the DUBOIS method (Dubois M et al., Analytical Chemistry, 28, 3, 350-356, 1956) and expressed as a percentage of the total dry matter content of the water lily extract. In some instances, the water lily extract may have a mineral fraction of between 20% and 60% (e.g., 25% to 50% or even 30% to 45%) by weight, based on the weight of the dry matter. Mineral fraction can by determined by weighing the crude ash residue resulting from incineration of the samples of the active ingredient at 550° C. in an electric muffle furnace.

It may be desirable to minimize or eliminate the amount of peptides/proteins and/or polyphenols present in the in the water lily extract, for example, to reduce the risk of an allergic response or other adverse reaction from a user. Accordingly, the water lily extracts herein may have a peptide fraction of less than 20% (e.g., less than 15%, 10%, or even less than 5%) by weight, based on the weight of the dry matter, and may be free of or substantially free of polyphenol compounds (i.e., less than 3%, 1%, 0.5%, or even less than 0.2%) by weight, based on the weight of the dry matter). The protein fraction of the water lily extract can be determined by the LOWRY method (Lowry et al., Protein measurement with the Folin reagent, J. Biol. Chem., 193, 265, 1951). In some instances, it may also be desirable to determine the size of the peptide compounds that are present, e.g., via FPLC type chromatography. The content of polyphenolic compounds can be determined according to the method provided in more detail below. The methods used to provide the peptide/protein and/or polyphenol levels in the water lily extract may be employed during the initial process of obtaining the water lily extract from the plant material and/or by subjecting the obtained water lily extract to further processing.

In some instances, the water lily extract may be mixed with other suitable materials (e.g., water, thickeners, humectants, solvents, preservatives, and/or solubilizers) prior to incorporation into a composition. For example, the water lily extract may be mixed with water or another suitable carrier (e.g., a polyhydric alcohol such as glycerin) to provide a mixture containing 0.001% to 50% (e.g., 0.01% to 40%, 0.1% to 20%, 0.5% to 10%, or even 1% to 5%) water lily extract. Water lily extract and/or a water lily mixture such as the one described above may be may be included in a composition at an amount of 0.0001% to 15%. 0.0002% to 10%, 0.001% to 15%, 0.025% to 10%, 0.05% to 10%, 0.05% to 5%, or even 0.1% to 5%, by weight, based on the weight of the composition.

Dermatologically Acceptable Carrier

The bilirubin degrading compositions herein include a dermatologically acceptable carrier (which may be referred to as a “carrier”). The phrase “dermatologically acceptable carrier” means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight.

The carrier can be in a wide variety of forms. In some instances, the solubility or dispersibility of the components (e.g., extracts, sunscreen active, additional components) may dictate the form and character of the carrier. Non-limiting examples include simple solutions (e.g., aqueous or anhydrous), dispersions, emulsions, and solid forms (e.g., gels, sticks, flowable solids, or amorphous materials). In some instances, the dermatologically acceptable carrier is in the form of an emulsion. The emulsion may have a continuous aqueous phase (e.g., an oil-in-water or water-in-oil-in-water emulsion) or a continuous oil phase (e.g., water-in-oil or oil-in-water-in-oil emulsion). The oil phase of the present invention may comprise silicone oils, non-silicone oils such as hydrocarbon oils, esters, ethers, and mixtures thereof. The aqueous phase typically comprises water and water-soluble ingredients (e.g., water-soluble moisturizing agents, conditioning agents, anti-microbials, humectants and/or other skin care actives). However, in some instances, the aqueous phase may comprise components other than water, including but not limited to water-soluble moisturizing agents, conditioning agents, anti-microbials, humectants and/or other water-soluble skin care actives. In some instances, the non-water component of the composition comprises a humectant such as glycerin and/or other polyol(s).

In some instances, the compositions herein are in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 30% by weight of composition (e.g., 1% to 20%, 2% to 15%, 3% to 12%, 4% to 10%, or even 5% to 8%) to help avoid some of the undesirable feel effects of oily compositions. The oil phase may include one or more volatile and/or non-volatile oils (e.g., botanical oils, silicone oils, and/or hydrocarbon oils). Some nonlimiting examples of oils that may be suitable for use in the present compositions are disclosed in U.S. Pat. No. 9,446,265 and U.S. Publication No. 2015/0196464.

The carrier may contain one or more dermatologically acceptable, hydrophilic diluents. As used herein, “diluent” includes materials in which the water lily extract can be dispersed, dissolved, or otherwise incorporated. Hydrophilic diluents include water, organic hydrophilic diluents such as lower monovalent alcohols (e.g., C₁-C₄) and low molecular weight glycols and polyols, including propylene glycol, polyethylene glycol (e.g., molecular weight of 200 to 600 g/mole), polypropylene glycol (e.g., molecular weight of 425 to 2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof.

Other Optional Ingredients.

The compositions herein may include one or more optional ingredients known for use in topical skin care compositions, provided that the optional components do not unacceptably alter the desired benefits of the composition. The additional ingredients should be suitable for use in contact with human skin tissue without undue toxicity, incompatibility, instability, allergic response, and the like. The optional components, when present, may be included at an amount of about 0.001% to 50% (e.g., 0.01% to 40%, 0.1% to 30%, 0.5% to 20%, or 1% to 10%), by weight of the composition. Some nonlimiting examples of additional ingredients include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunscreen agents, sunless tanning agents, lubricants, anti-acne agents, anti-cellulite agents, chelating agents, anti-wrinkle actives, anti-atrophy actives, phytosterols and/or plant hormones, N-acyl amino acid compounds, antimicrobials, and antifungals. Other non-limiting examples of additional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/00092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

Conditioning Agents

The compositions herein may include 0.1% to 50% by weight of a conditioning agent (e.g., 0.5% to 30%, 1% to 20%, or even 2% to 15%). Adding a conditioning agent can help provide the composition with desirable feel properties (e.g., a silky, lubricious feel upon application). Some non-limiting examples of conditioning agents include, hydrocarbon oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, lanolin, wax esters, beeswax derivatives, sterols and phospholipids, salts, isomers and derivatives thereof, and combinations thereof. Particularly suitable examples of conditioning agents include volatile or non-volatile silicone fluids such as dimethicone copolyol, dimethylpolysiloxane, diethylpolysiloxane, mixed C1-30 alkyl polysiloxanes, phenyl dimethicone, dimethiconol, dimethicone, dimethiconol, silicone crosspolymers, and combinations thereof. Dimethicone may be especially suitable, since some consumers associate the feel properties provided by certain dimethicone fluids with good moisturization. Other examples of silicone fluids that may be suitable for use as conditioning agents are described in U.S. Pat. No. 5,011,681.

Rheology Modifiers

The compositions herein may include 0.1% to 5% of a rheology modifier (e.g., thickening agent) to provide the composition with suitable rheological and skin feels properties. Some non-limiting examples of thickening agents include crosslinked polyacrylate polymers, polyacrylamide polymers, polysaccharides, gums and mixtures thereof. In a particularly suitable example, the composition may include a superabsorbent polymer thickening agent such as sodium polyacrylate, starch grafted sodium polyacrylate, or a combination of these. Some non-limiting examples of superabsorbent polymer thickeners are described in, for example, U.S. Pat. No. 9,795,552.

Some consumers find compositions that use silicone fluids as conditioning agents to be undesirably greasy or heavy feeling. Thus, it may be desirable to provide a composition that is free of or substantially free of silicone fluid. It may also be desirable to tailor a superabsorbent polymer thickener to provide the composition with a light, airy feel, for example, by adjusting the amount of water in the composition, the water:oil ratio (e.g., 12:1 to 1:1), and/or the ratio of water to thickener or oil to thickener.

Emulsifiers

When the composition is in the form of an emulsion, it may contain an emulsifier. Emulsifiers may be nonionic, anionic or cationic. Suitable emulsifiers are disclosed in, for example, U.S. Pat. Nos. 3,755,560, 4,421,769, U.S. Publication No. 2006/0275237 and McCutcheon's Detergents and Emulsifiers, North American Edition, pages 317-324 (1986). Suitable emulsions may have a wide range of viscosities, depending on the desired product form.

When including optional ingredients in the compositions herein, it may be desirable to select ingredients that do not form complexes or otherwise undesirably interact with other ingredients in the composition at low pH, especially pH sensitive ingredients like niacinamide, salicylates and peptides (e.g., palmitoyl-lysine-threonine (pal-KT) or palmitoyl-lysine-threonine-threonine-lysine-serine (pal-KTTKS). In some instances, it may be desirable to select skin care actives that function via different biological pathways so that the actives do not interfere with one another, which could reduce the efficacy of both agents.

Method of Use

The present method includes identifying a target portion of skin on a person in need of treatment and applying a composition comprising an effective amount of water lily extract, and optionally one or more additional skin care agents, to the target portion of skin. The target portion of skin may be on a facial skin surface such as the forehead, perioral, chin, periorbital, nose, and/or cheek) or another part of the body (e.g., hands, arms, legs, back, chest). The person in need of treatment can be one who exhibits an undesirable level of bilirubin in their skin and/or exhibits another undesirable cosmetic skin condition. Bilirubin level may be determined according to any suitable method known in the art. For example, bilirubin level may be determined by a blood sample analysis. In another example, an undesirable bilirubin level may be indicated if the target portion of skin has a b* value greater than a predetermined threshold level corresponding to an undesirably high bilirubin level. The b* value may be determined according to the color imaging method described in more detail below. In some instances, a person may be identified as being in need of treatment when their skin exhibits a yellow or sallow appearance and/or the person has an uneven skin tone. In another example, a person in need of treatment may be identified when an undesirable level of yellowness is determined to be present in a target portion of skin by an expert (e.g., dermatologist or cosmetologist). The person in need of treatment may also identify the target portion of skin when bruising is present. In some instances, a target portion of skin may not appear to be suffering from a buildup of bilirubin, but a user (e.g., a person suffering from or prone to jaundice or bruising) may still wish to treat the target portion of skin as a preventative measure (e.g., if the person is prone to conditions that cause bilirubin buildup such as jaundice).

The composition may be applied to a target portion of skin and, if desired, to the surrounding skin at least once a day, twice a day, or on a more frequent daily basis, during a treatment period. When applied twice daily, the first and second applications are separated by at least 1 to 12 hours. Typically, the composition is applied in the morning and/or in the evening before bed. When used according to the methods herein, the present compositions improve the appearance of skin by reducing bilirubin level, as demonstrated by a reduction in b* of at least 5% (e.g., at least 10%, 15%, 20%, 25%, or more), according to the method hereinbelow. In some instances, a reduction in bilirubin level may be determined by measuring bilirubin levels according to a conventional in vivo method (e.g., blood analysis) and comparing the measured level to a predetermined threshold value or a level of bilirubin measured prior to the beginning of the treatment period.

The treatment period is ideally of sufficient time for the water lily extract present in the composition to reduce the bilirubin level of a target portion of skin. In some instances, the bilirubin reduction benefit provided by the water lily extract may be demonstrated by a reduction in b* value relative to a predetermined b* value (i.e., a b* value determined prior to the beginning of the treatment period). Additionally or alternatively, the bilirubin reduction benefit may be demonstrated by comparing a measured b* value and/or bilirubin level to a control value (e.g., vehicle control) or other reference value (e.g., an identical composition except with a different water lily extract). The treatment period may last for at least 1 week (e.g., about 2 weeks, 4 weeks, 8 weeks, or even 12 weeks). In some instances, the treatment period will extend over multiple months (i.e., 3-12 months). In some instances, the composition may be applied most days of the week (e.g., at least 4, 5 or 6 days a week), at least once a day or even twice a day during a treatment period of at least 2 weeks, 4 weeks, 8 weeks, or 12 weeks.

The step of applying the composition may be accomplished by localized application. In reference to application of the composition, the terms “localized”, “local”, or “locally” mean that the composition is delivered to the targeted area (e.g., a hyperpigmented spot or portion thereof) while minimizing delivery to skin surfaces where treatment is not desired. The composition may be applied and lightly massaged into an area of skin. The form of the composition or the dermatologically acceptable carrier should be selected to facilitate localized application. While certain embodiments herein contemplate applying a composition locally to an area, it will be appreciated that compositions herein can be applied more generally or broadly to one or more skin surfaces. In certain embodiments, the compositions herein may be used as part of a multi-step beauty regimen, wherein the present composition may be applied before and/or after one or more other compositions.

### Methods

Phenolic Compound Assay

This method provides a suitable means to determine the amount of polyphenolic compounds in the water lily extract. The polyphenolic compounds form, in the presence of potassium ferricyanide, colored compounds, detectable at 715 nm. The coloring intensity is proportional to the quantity of polyphenolic compounds. Readings are taken from a standard sample of gallic acid ranging between 40 and 120 mg/l. The results obtained for the samples allow a straight-line optical density to be traced as a function of the concentration and the polyphenols level of the samples is read directly on this straight line. The content of polyphenolic compounds of the hydrolysate according to the invention may be expressed as a percentage of gallic acid equivalent relative to the dry matter content of the water lily extract.

Bilirubin Degradation Assay

This assay provides a method of measuring the ability of a material to degrade bilirubin. Three replicates of each test sample are prepared in a 96-well plate (e.g., a FALCON brand 96-well tissue culture plate or equivalent) at a total volume of 250 μl/well. A stock of 250 ug/ml indirect bilirubin (i.e., the unconjugated form of bilirubin most commonly found in blood serum) is made by dissolving bilirubin powder (Cayman Chemicals Company, Catalog #17161) in DMSO (Sigma, Catalog #D8414-100 ml) to yield a stock solution at 10× working concentration. Working concentration of bilirubin is set at 25 ug/ml for every well except negative/vehicle control wells. The experimental set up typically includes positive control wells that contain 25 ug/ml bilirubin, 225 ul PBS, and 10% DMSO. The positive control wells may be prepared by mixing 25 ul of 250 ug/ml bilirubin stock solution and 225 ul PBS (AccuGENE, Catalog #51225). For negative/vehicle control wells, 25 ul DMSO is used in place of bilirubin. Test sample wells contain 25 ul of 250 ug/ml bilirubin stock solution, 200 ul PBS and 25 ul of each treatment solution made as 10× concentrate stock, e.g., for a 0.1% working concentration of water lily extract, a OX concentrate stock is made at 1%.

Bilirubin is unstable when exposed to ambient light. To avoid degradation from ambient light exposure, the plate(s) containing the test samples are covered with aluminum foil. The covered plates are then placed on top of a microplate shaker (VWR, Catalog #12620-938). Incubation was carried out at room temperature for 23-hr with constant shaking at 150 rpm. Bilirubin concentration was quantified after 23-hr incubation to determine the effect of bilirubin degradation as a result of active treatment in comparison to positive bilirubin control.

Bilirubin Quantitation

The bilirubin standards and the samples were analyzed using gradient high-performance liquid chromatography with tandem mass spectrometry (HPLC-MS/MS). Bilirubin can be nicely separated by a reversed-phase column (ATLANTIS T3 Column, 100 angstrom, 3 μm, 2.1 mm×50 mm available from Waters Corporation, Milford, Mass.). Bilirubin and the corresponding stable isotope labeled internal standard (ISTD) were monitored by electrospray ionization (ESI) in positive mode using the selected-reaction-monitoring schemes shown in Table 1. The ISTD used in this invention is d4-bilirubin (Toronto Research Chemicals, North York, ON, Canada) with four deuterium labeled. A standard curve was constructed by plotting the signal, defined here as the peak area ratio (peak area analyte/peak area ISTD), for each standard versus the concentration of each analyte for the corresponding standard. The concentration of bilirubin in the calibration standards and samples were then back-calculated using the generated regression equation.

TABLE 1
Multiple Reaction Monitoring (MRM) transitions for analytes and
their corresponding stable isotope labeled internal standards

| Analytes | MRM | Internal Standards | MRM |
|---|---|---|---|
| bilirubin | 585.2 → 299.2 | d4-bilirubin | 589.2 → 301.2 |

### EXAMPLES

### Example 1: Formulations

Table 2 below provides examples of topical skin care compositions comprising water lily extract. The exemplary compositions are made by blending the A phase components with a suitable mixer (e.g., Tekmar RW20DZM or equivalent) and heating to a temperature of 70-80° C. and maintaining the temperature while stirring. Separately, the B phase components are blended with a suitable mixer and heated to 70-75° C., while maintaining temperature during mixing. Phase B is added to Phase A while mixing well to form an oil-in-water (O/W) emulsion. The emulsion is then milled using a suitable mill (e.g., Tekmar T-25 or equivalent) for 5 minutes. When the emulsion is at 60° C., phase C is added while continuing to mix. At 40° C., the ingredients of phase D and E are added to the emulsion. The emulsion is then milled for 5 minutes to provide a uniform composition.

TABLE 2

| Component | I | II | III | IV | V | VI | VII | VIII | IX |
|---|---|---|---|---|---|---|---|---|---|
| Phase A | | | | | | | | | |
| Water | qs | qs | qs | qs | qs | qs | qs | qs | qs |
| Glycerol | 5.00 | 7.00 | 3.00 | 15.0 | 7.00 | 5.00 | 5.00 | 3.00 | 5.00 |
| Disodium EDTA | 0.10 | 0.05 | 0.10 | 0.10 | 0.05 | 0.05 | 0.05 | 0.05 | 0.10 |
| Phase B | | | | | | | | | |
| Dimethicone 5 cSt | — | — | — | — | — | — | — | 10.0 | 15.0 |
| Dimethicone and Dimethicone | — | — | — | — | — | — | — | 13.0 | 15.0 |
| Crosspolymer | | | | | | | | | |
| Laureth-4 | — | — | — | — | — | — | — | 0.25 | 0.35 |
| Polysorbate 20 | — | — | — | — | — | — | — | 0.15 | 0.25 |
| Tapioca Starch and | — | — | — | — | — | — | — | 2.50 | 3.50 |
| Polymethylsilsesquioxane | | | | | | | | | |
| Avobenzone | — | — | — | 3.00 | — | 3.00 | — | — | — |
| Homosalate | — | — | — | 15.0 | — | 10.0 | — | — | — |
| Octisalate | — | — | — | 5.00 | — | 5.00 | — | — | — |
| Octocrylene | — | — | — | 2.60 | — | 9.00 | — | — | — |
| Isopropyl Isostearate | 5.00 | 2.50 | 1.00 | — | — | — | — | — | — |
| Isohexadecane | 1.00 | 1.50 | 3.00 | — | — | — | — | — | — |
| Cetyl Alcohol | 0.25 | 0.50 | 0.32 | 0.40 | 0.40 | 0.30 | 0.50 | — | — |
| Tocopherol Acetate | | 0.50 | 0.25 | 1.00 | 0.25 | 0.25 | 0.25 | — | — |
| PEG-100 Stearate | 0.20 | 0.10 | 0.10 | 0.30 | 0.10 | 0.20 | 0.10 | — | — |
| Stearyl Alcohol | 0.50 | 1.50 | 0.40 | 0.60 | 0.50 | 0.40 | 0.60 | — | — |
| Behenyl Alcohol | 0.40 | 1.00 | 0.50 | 0.50 | 0.40 | 0.35 | 0.50 | — | — |
| Ethyl Paraben | 0.20 | 0.15 | 0.20 | 0.25 | — | — | — | — | — |
| Propyl Paraben | 0.10 | 0.15 | 0.10 | 0.15 | — | — | — | — | — |
| Polymethylsilsesquioxane | 1.25 | 2.50 | 1.00 | — | — | — | — | — | — |
| Phase C | | | | | | | | — | — |
| Titanium Dioxide | — | 0.50 | — | 0.25 | — | — | — | — | — |
| Tapioca Starch and | — | — | — | — | — | 12.0 | | — | — |
| Polymethylsilsesquioxane | | | | | | | | | |
| Vinyl Dimethicone/Methicone | 1.50 | | 1.50 | 3.50 | 5.00 | — | 7.50 | — | — |
| Silsesquioxane Crosspolymer | | | | | | | | | |
| Sodium Polyacrylate Starch | — | — | — | — | 1.50 | 1.00 | 1.50 | — | — |
| Hydroxyethyl acrylate/sodium | 2.00 | 1.50 | 2.50 | 2.00 | — | — | — | 1.25 | 2.00 |
| acryloyldimethyltaurate copolymer | | | | | | | | | |
| Phase D | | | | | | | | | |
| Water | 5.00 | 10.0 | 10.0 | 5.00 | 10.0 | 10.0 | 10.0 | 5.00 | 10.0 |
| Water Lily Extract | 0.25 | 0.40 | 3.50 | 0.40 | 10.0 | 1.25 | 1.00 | 1.00 | 5.00 |
| Niacinamide | 1.00 | | 3.50 | | 2.00 | 5.00 | | — | 1.00 |
| Dexpanthenol | 0.25 | 0.50 | 0.50 | 1.00 | 1.00 | 1.50 | 0.25 | 1.00 | 0.50 |
| Phase E | | | | | | | | | |
| Benzyl alcohol | 0.25 | 0.40 | 0.25 | 0.50 | — | — | — | — | — |
| Hexanediol and Caprylyl Glycol | — | — | — | — | 0.70 | 0.80 | 0.70 | 0.70 | 1.00 |
| Phenoxyethanol | — | — | — | — | 0.3 | 0.4 | 0.5 | 0.20 | 0.25 |
| Dimethicone/dimethiconol | 0.5 | 1.00 | 2.00 | 1.00 | 2.00 | 2.00 | 1.00 | 1.75 | 1.00 |

### Example 2: Liquid Water Lily Extract

This example describes a process for providing a suitable water lily extract for use in the cosmetic compositions herein. In this example, flowers of the Nymphaea alba plant are dried and then ground into a powder. The dried powder is solubilized in water (100 g/L) and subjected to enzymatic hydrolysis with a carbohydrase (e.g., cellulase, glucanase, pectinase, xylanase, arabinose or a combination of these) and a protease (e.g., endoprotease, cysteinprotease, exopeptidase, aminopeptidase, metalloprotease or a combination of these). The soluble (i.e., aqueous) and insoluble phases are separated and the insoluble phase can be discarded. The soluble phase is thermally treated to inactivate any enzymes that may be present. The solution is then clarified and purified. The concentration of the clarified and purified solution can be adjusted to the desired level by adding or removing water. The solution is then filtered and sterilized to provide the water lily extract for incorporation into a product. Table 3 below lists a non-limiting example of physical characteristics of the resulting water lily extract. The amounts of polyphenol, carbohydrate, protein and mineral shown in Table 2 are all weight percentages based on the weight of the dry matter.

TABLE 3

| Property | Value |
|---|---|
| Dry Matter content | 29 g/L |
| Polyphenol content | <0.2% (threshold detection) |
| Carbohydrate content | 51% |
| Protein content | 10% |
| Mineral content | 39% |

### Example 3: Clinical Study

In this study, a skin care composition comprising an effective amount of water lily extract was tested to evaluate its ability to improve the appearance of skin relative to a placebo (i.e., an identical composition, except without the water lily extract). The test composition, shown below in Table 4, was made using conventional methods. The test composition was evaluated for its ability to improve a variety of cosmetic skin conditions (i.e., skin radiance, skin texture, skin brightness, and the appearance of fine lines and wrinkles). The study was conducted on 19 healthy female test subjects aged 35 to 55 years, with an average age of 43±6 years. The subjects were asked to apply the test composition to half of their face and the placebo to the other half of their face twice per day for 28 days. For skin brightness/lightness, the test subjects were evaluated by expert graders based on four categories: skin grain, transparency, radiance and light pink color. The test composition provided statistically significant improvement in all 4 categories versus the placebo. The test subjects were also asked to self-evaluate via a questionnaire. More than 70% of the test subjects indicated that the test composition improved skin brightness, radiance, texture, even complexion, and appearance of wrinkles versus the placebo. In other testing, the test composition demonstrated the ability to improve skin smoothness as determined using an interferometry technique. The test composition also demonstrated the ability to act as an antioxidant against UV induced oxidative stress.

TABLE 4

| Ingredient | Wt % |
|---|---|
| Isononyl isononanoate (Lanol 99, Seppic) | 5.0% |
| Water lily hydrolysate | 3.0% |
| Arachidyl alcohol/Behenyl Alcohol/ | 3.0% |
| Arachidyl glucoside (Montanov 202, Seppic) | |
| Cetearyl alcohol/Cetearyl glucoside | 2.0% |
| (Montanov 68, Seppic) | |
| Preservatives | 0.7% |
| Polyacrylamide/C13-14 isoparaffin/ | 0.3% |
| Laureth-7 (Sepigel 305, Seppic) | |
| Water | qsp 100% |

### Example 4: Comparative Testing

This example demonstrates the unexpected bilirubin degradation benefit of the present water lily extract by comparing it to commercially available water lily extracts. The water lily extract material used to make the inventive example was obtained from Silab, S.A., and contains Nymphaea alba flower extract. The water lily extract material used in the inventive example is not commercially available. Comparative example A was made from a water lily extract material commercially available from IFF/Lucas Meyer Cosmetics, Quebec, as Hydralphatine™ Asia and contains Nymphaea alba root extract. Comparative Example B was made from a water lily extract material commercially available from Seppic, S.A., France, as Sepicalm™ VG WP and contains Nymphaea alba flower extract. Comparative Example C was made from a water lily extract material commercially available from GFC Life Science Co., Ltd., Korea, as Phyto-G™ and contains Nymphaea alba flower extract. A positive control leg is provided by mixing 25 ug/ml indirect bilirubin with PBS buffer resulting in DMSO being 10% in final reaction mixture. A solution of 10% DMSO in PBS is used as the negative (vehicle) control.

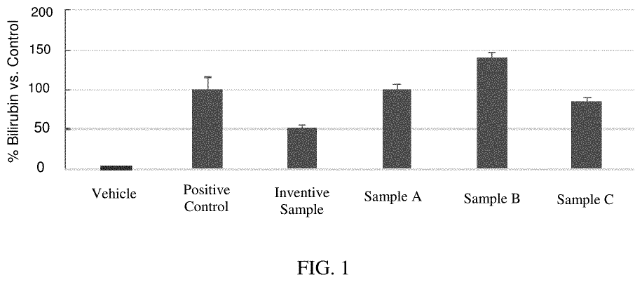

Testing was done in triplicate by mixing each water lily extract material at 0.4% v/v (1:250 dilution of the original material) with 25 ug/ml indirect bilirubin in PBS buffer in 96-well plate. The final reaction mixture has 250 ul volume for each testing well that contains 10% DMSO. Each plate is covered with aluminum foil and incubated at room temperature for twenty-three hours on an orbital shaker set at 150 rpm. After incubation, the final reaction mixture is mixed well by pipetting up and down 3 times followed by dilution of each sample at 1:50 in 0.1% triethanolamine. 150 ul of each diluted sample is aliquoted into an HPLC vial and quantified using multiple reaction monitoring mass spectroscopy (MRM). Quantified bilirubin level of each sample is normalized vs. the mean level of the positive control leg. A t-test versus the bilirubin positive control leg is conducted to determine statistical significance. The results of the test are summarized in Table 5 below and illustrated in FIG. 1. The bilirubin values provided in Table 5 are averages of the triplicate samples of the corresponding test leg. A p-value of 0.1 or less is considered significant.

TABLE 5

| | % Bilirubin quant | |
|---|---|---|
| Sample (n = 3) | vs. control | p-value |
| Vehicle Control | 0 | 0.000 |
| Positive Control | 100 | 1.000 |
| Comparative Example A: | 100 | 0.812 |
| 0.4% (v/v) water lily extract¹ | | |
| Comparative Example B: | 140 | 0.013 |
| 0.4% (v/v) water lily extract² | | |
| Comparative Example C: | 85 | 0.166 |
| 0.4% (v/v) water lily extract³ | | |
| Inventive Example: | 52 | 0.005 |
| 0.4% (v/v) water lily extract | | |

¹Hydralphatine ™ Asia available from IFF/Lucas Meyer Cosmetics, Quebec.
²Sepicalm ™ VG WP available from Seppic, S.A., France.
³Phyto-G ™ available from GFC Life Science Co., Ltd., Korea.

As illustrated in Table 5 and FIG. 1, the Inventive Example significantly reduces bilirubin levels relative to the positive control. However, the bilirubin levels observed for Comparative Examples A and C are not significantly different from the positive control. Comparative Example C appears to actually increase bilirubin levels. The apparent increase in Example C is believed to be a result of the natural degradation process of bilirubin. Indirect bilirubin is relatively unstable and degrades naturally over time. However, other ingredients in Comparative Example C (a commercially available product) may be slowing down the natural bilirubin degradation process relative to the reference leg, which manifests as a higher bilirubin quantity in the assay.

As can be seen from this example, selecting the right water lily extract provides an unexpected bilirubin degradation benefit, which may in turn improve the appearance of sallow-looking skin caused by bilirubin buildup in skin tissue.

### Example 5: Correlating Bilirubin Concentration to Yellowness

It is believed, without being limited by theory, that bilirubin concentration in skin has a direct correlation to a yellow or sallow appearance. To determine the correlation between bilirubin concentration and yellowness, a bilirubin standard curve is generated. Bilirubin powder (Cayman Chemicals Company, Catalog #17161) is dissolved in DMSO (Sigma, Catalog #D8414-100 ml) to make a 500 ug/ml bilirubin stock. This bilirubin stock is diluted in PBS as shown in Table 1 above to yield bilirubin levels at 50, 25, 12.5, 5, 2.5, and 0 ug/ml. To measure yellowness (b*), the samples are loaded into a 96-well plate in triplicate and the absorbance of each sample is measured in 10 nm increments from 350 nm to 750 nm using a suitable spectrophotometer. The absorbance spectrum from the yellowness measurement are then converted to L*a*b* values by a computer using suitable conversion software.

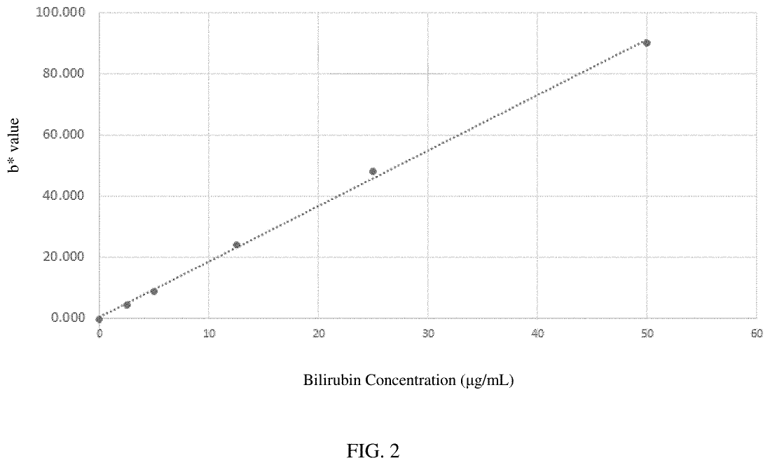

As shown in Table 6 and FIG. 2, the b* scores show a linear correlation to bilirubin concentration in the range of 0 ug/ml to 50 ug/ml bilirubin. Human biological bilirubin concentration falls within this concentration range. Thus, the data show that bilirubin concentration in skin has a direct correlation to a sallow appearance.

TABLE 6

| Bilirubin Concentration (μg/mL) | Average b* value |
|---|---|
| 0 | −0.417 |
| 2.5 | 4.258 |
| 5 | 8.582 |
| 12.5 | 23.889 |
| 25 | 47.993 |
| 50 | 90.017 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method of improving the appearance of skin, comprising:
a) identifying a target portion of skin where treatment is desired; and
b) applying a composition comprising an effective amount of water lily extract to the target portion of skin during a treatment period, wherein the effective amount of water lily extract reduces bilirubin level.

2. The method of claim 1, wherein the water lily extract comprises a hydrolysate.

3. The method of claim 1, wherein the composition comprises about 0.00001% to about 10% water lily extract, by weight, based on the weight of the composition.

4. The method of claim 1, wherein the water lily extract is obtained from a flower of Nymphaea alba.

5. The method of claim 1, wherein the target portion of skin exhibits a sign of bilirubin accumulation.

6. The method of claim 1, wherein the effective amount of water lily extract reduces bilirubin level by at least 10%, according to the Bilirubin Degradation Assay.

7. The method of claim 6, wherein the additional ingredient includes a superabsorbent polymer thickening agent selected from sodium polyacrylate, starch grafted sodium polyacrylate, and combinations thereof.

8. The method of claim 6, wherein the composition includes at least about 10% of a silicone fluid.

9. A skin care composition, comprising:
a) an effective amount of Nymphaea alba extract, wherein the effective amount of Nymphaea alba reduces bilirubin level according to the Bilirubin Degradation Assay;
b) a dermatologically acceptable carrier; and
c) an added preservative.

10. The composition of claim 9, wherein the Nymphaea alba extract comprises a hydrolysate.

11. The skin cam composition of claim 9, wherein the Nymphaea alba extract is present at about 0.001% to about 10% by weight of the composition.

12. The skin cam composition of claim 9, wherein the Nymphaea alba extract is an aqueous solution with a dry matter content of about 10 g/L to about 50 g/L.

13. The skin care composition of claim 12, wherein the Nymphaea alba extract comprises about 20% to about 70% carbohydrate based on the weight of the dry matter.

14. The skin care composition of claim 12, wherein the Nymphaea alba extract comprises about 20% to about 60% mineral based on the weight of the dry matter.

15. The skin care composition of claim 12, wherein the Nymphaea alba extract comprises less than about 20% protein, based on the weight of the dry matter.

16. The skin care composition of claim 12, wherein the Nymphaea alba extract comprises less than about 3% polyphenol based on the weight of the dry matter.

17. The skin care composition of claim 9, wherein the composition further comprises at least one additional ingredient selected from the group consisting of vitamins, minerals, peptides, sugar amines, sunscreen agents, oil control agents, flavonoid compounds, anti-oxidants, preservatives, protease inhibitors, tyrosinase inhibitors, anti-inflammatory agents, moisturizing agents, exfoliating agents, skin lightening agents, lubricants, anti-acne actives, chelating agents, anti-wrinkle actives, anti-atrophy actives, phytosterols, N-acyl amino acid compounds, antimicrobials, and antifugals, conditioning agents, emulsifiers, rheology modifiers, and combinations of these.

18. The skin care composition of claim 17, wherein the additional ingredient is selected from the group consisting of vitamin B3 compounds, vitamin E compounds, peptides, retinoids and combinations thereof.

19. The skin cam composition of claim 17, wherein the additional ingredient includes a superabsorbent polymer thickening agent selected from sodium polyacrylate, starch grafted sodium polyacrylate, and combinations thereof.

20. The skin cam composition of claim 9, further comprising at least about 10% of a silicone fluid.
